(19) United States
(12) Patent Application Publication
(10) Pub. No.: US 2008/0081357 A1
(43) Pub. Date: Apr. 3, 2008

(54) METHOD AND APPARATUS FOR ISOLATING NUCLEIC ACIDS FROM A CELL USING CARBON NANOTUBES AND SILICA BEADS

(75) Inventors: Young-nam KWON, Yongin-si (KR); Jeong-gun LEE, Yongin-si (KR); Jeong-hee LEE, Yongin-si (KR); Ju-chul PARK, Yongin-si (KR); Chang-eun YOO, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/733,846

(22) Filed: Apr. 11, 2007

(30) Foreign Application Priority Data
Sep. 29, 2006 (KR) 10-2006-0096288

(51) Int. Cl. C12P 19/34; C12M 1/42; C12N 13/00

(52) U.S. Cl. 435/91.2; 435/173.7; 435/306.1

(57) ABSTRACT

Provided herein are a method and an apparatus for isolating nucleic acids from cells. The method comprises introducing carbon nanotubes (CNTs) and silica beads into a solution containing the cells, irradiating the solution with a laser beam disrupt the cells releasing the nucleic acids from the disrupted cells, thereby binding the nucleic acids to the silica beads, and adding a nucleic acid eluting solution to the silica beads to which the nucleic acids are bound, to elute the nucleic acids from the silica beads.

This application claims priority to Korean Patent Application No. 10-2006-0096288, filed on Sep. 29, 2006, and all the benefits accruing therefrom under 35 U.S.C. § 119, the disclosure of which is incorporated herein in its entirety by reference.

### BACKGROUND OF THE INVENTION

1. Field of the Invention

The present invention relates to a method and apparatus for isolating nucleic acids from cells using carbon nanotubes (CNTs), silica beads, and a laser.

2. Description of the Related Art

In general, the molecular diagnosis of pathogens consists of four steps, i.e., cell lysis, DNA isolation, DNA amplification, and DNA detection.

Further, efficient extraction of DNA from cells is needed in a variety of applications, and inter alia, such extraction of DNA is essential in molecular diagnosis, particularly for the identification and quantification of pathogenic bacteria. Molecular diagnosis is generally performed by DNA extraction followed by DNA amplification.

Cell lysis is conventionally performed using a mechanical, chemical, thermal, electrical, ultrasonic or microwave method (Michael T. Taylor et al., Anal. Chem., 73, 492-496 (2001)).

Chemical methods for cell lysis involve the use of a lysing agent for disrupting cells and releasing DNA. Further, additional treatment of cell extract is required using a chaotropic reagent to denature proteins in the cell extract. One disadvantage with the chemical methods for cell lysis is that harsh chemicals are used to disrupt cells. Such chemicals may impede a PCR reaction that is performed using the cell extract after the cell lysis, and thus, purification of the DNA from the cell extract is necessary before performing the PCR reaction. Furthermore, chemical methods for cell lysis are labor-intensive, time-consuming and costly, and often produce low DNA recovery yields.

Thermal methods for cell lysis involve repeated freeze-thaw cycles. One disadvantage with the thermal method is that the method is often unable to disrupt many intracellular structures. Heating is an alternative method of disrupting the cell walls or cell membranes. One disadvantage with such a method is that heating causes denaturation of proteins, which may adhere to the released DNA, and thereby hinder DNA amplification.

The ultrasonic method is an alternative physical method for disrupting cells and releasing DNA. For the ultrasonic method, a cell solution or a cell suspension is placed in the chamber of an ultrasonic water bath. Ultrasonic cell destruction is highly ineffective in cell lysis. First, the energy distribution of an ultrasound is not uniform, and such non-uniform distribution of ultrasonic energy induces results that lack consistency. Further, the ultrasonic water bath is incapable of concentrating the ultrasonic energy into the cell solution container, and it usually takes several minutes to achieve complete disruption of the cells. Finally, ultrasonic cell destruction produces a sound that is unpleasant to human ears.

An alternative method for disrupting cells and releasing DNA employs a laser. The use of a laser to disrupt cells has many advantages and is highly applicable to a lab-on-a-chip (LOC) (Huaina Li et al., Anal Chem, 73, 4625-4631 (2001)).

U.S. Patent Publication No. 2003/96429 A1 discloses a laser-induced cell lysis system. According to this publication, efficient cell lysis does not occur when only a laser beam is used.

U.S. Pat. No. 6,685,730 discloses optically absorbing nanoparticles to enhance tissue repair. This patent describes a method of joining a tissue comprising delivering nanoparticles having a diameter of 1-1000 nanometers that absorb light at one or more wavelengths to the tissue to be joined, and exposing said nanoparticles to light at one or more wavelengths that are absorbed by the nanoparticles. This method causes only a loss of function of cells by using the laser beam and the nanoparticles.

In an attempt to develop an efficient method for isolating nucleic acids from cells, the inventors studied methods of disrupting cells using a laser. The inventors developed a novel method for disrupting cells utilizing a laser beam, allowing for the release of DNA from the cell. Nucleic acids released from the disrupted cells can be isolated using silica beads, and as a result, the nucleic acids can be efficiently isolated from the cells.

### BRIEF SUMMARY OF THE INVENTION

The present invention provides a method of rapidly isolating nucleic acids from cells using carbon nanotubes (CNTs), silica beads, and a laser.

The present invention also provides an apparatus for continuously performing isolation and amplification of nucleic acids in a rapid manner, comprising a cell or virus disruption micro-chamber, a sample storage unit, a laser generation unit, a polymerase chain reaction (PCR) mixture storage unit, and a heating and cooling unit.

In one embodiment, the present invention provides a method of isolating nucleic acids from cells, comprising introducing carbon nanotubes (CNTs) and silica beads into a sample solution containing the cells, irradiating the sample solution with a laser beam to disrupt the cells releasing nucleic acids from the disrupted cells, wherein the nucleic acids bind to the silica beads, and introducing a nucleic acid-eluting solution to the silica beads to which the nucleic acids are bound, to elute the nucleic acids from the silica beads.

In another embodiment, the present invention is directed to an apparatus for continuously performing isolation and amplification of nucleic acids, comprising a cell disruption micro-chamber having a sample inlet through which a sample solution containing cells, CNTs, and silica beads are introduced; a sample storage unit being in a fluid communication with the cell disruption micro-chamber through a micro-channel and supplying the sample solution containing the cells, CNTs, and silica beads to the cell disruption micro-chamber through the micro-channel; and a laser generation unit attached to the cell disruption micro-chamber wherein the laser generation unit can irradiate the cell disruption micro-chamber with a laser beam; and a polymerase chain reaction (PCR) mixture storage unit being in a fluid communication with the cell disruption micro-chamber through a micro-channel, wherein the polymerase chain reaction (PCR) mixture storage unit can supply a PCR mixture to the cell disruption micro-chamber through the micro-channel; and a heating and cooling unit, wherein the heating and cooling unit can heat or cool the cell disruption micro-chamber.

In another embodiment, the present invention provides a lab-on-a-chip comprising the apparatus for continuously performing isolation and amplification of nucleic acids.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram illustrating a method of isolating nucleic acids from cells according to an embodiment of the present invention;

FIG. 2 is schematic diagram illustrating a silica bead used in the method of isolating nucleic acids from cells described with reference to FIG. 1;

FIG. 3 provides photographic representations of transmission electron microscope (TEM) images of Pt-impregnated carbon nanotubes (CNTs);

FIG. 4 provides photographic representations of atomic force microscope (AFM) images of Pt-impregnated CNTs;

FIG. 5 provides photographic representations of TEM images of bare CNTs;

FIG. 6 provides photographic representations of AMF images of bare CNTs;

FIG. 7 is a graph illustrating laser irradiation time vs. temperature of a solution; and

FIG. 8 is a graph illustrating the number of E. coli cells after laser irradiation of a sample with or without CNTs.

### DETAILED DESCRIPTION OF THE INVENTION

The invention will now be described more fully hereinafter with reference to the accompanying drawings, in which embodiments of the invention are shown. The invention may, however, be embodied in many different forms and should not be construed as limited to embodiments set forth herein. Rather these embodiments are provided so that this disclosure will be through and complete, and will fully convey the scope of the invention to those skilled in the art.

In one embodiment of the present invention, there is provided a method of isolating nucleic acids from cells.

As used herein, the term “cell” means a prokaryotic or eukaryotic cell, a plant cell, a bacteria cell, a pathogenic cell, a yeast cell, an aggregate of cells, a virus, a fungus, or other nucleic acid containing biological material, such as, for example, an organelle.

As used herein, the term “nucleic acid” means DNA or RNA, or a combination of both. The DNA or RNA can be in any possible configuration, i.e., in the form of double-stranded (ds) nucleic acid, or in the form of single-stranded (ss) nucleic acid, or as a combination thereof (in part ds or ss).

As used herein, the term “sample solution” is a sample that comprises or is formed of a cell or tissue, such as a cell or biological liquid isolated from an animal or plant containing nucleic acids. The sample solution can be any solution having nucleic acids, such as animal cells, plant cells, bacteria, viruses, or phages. In one advantageous embodiment, the animal can be a human. The biological sample can be saliva, sputum, blood, blood cells (for example, red blood cells or white blood cells), amniotic fluid, serum, semen, bone marrow, tissue or a micro needle biopsy sample, urine, peritoneum fluid, pleura fluid, or cell cultures. In addition, the biological sample can be a tissue section, such as a frozen section taken for a histological object. Preferably, the biological sample is a clinical sample obtained from a human patient. More preferably, the biological sample is blood, urine, saliva, or sputum. Furthermore, the term “biological sample” means a sample that is formed comprising an organism, group of organisms from the same or different species, cells or tissues, obtained from the environment, such as from a body of water, from the soil, or from a food source or an industrial source.

In one embodiment, the method comprises introducing carbon nanotubes (CNTs) and silica beads into a sample solution containing the cells; irradiating the sample solution with a laser beam to disrupt the cells and release the nucleic acids from the disrupted cells, wherein the nucleic acids bind to the silica beads; and introducing a nucleic acid eluting solution to the silica beads to which the nucleic acids are bound, to elute the nucleic acids from the silica beads.

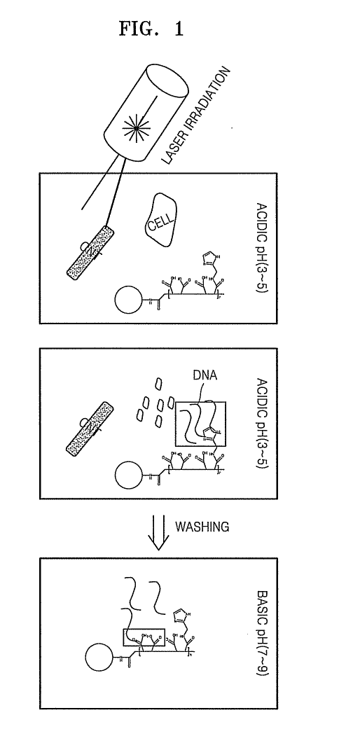

FIG. 1 is a schematic diagram illustrating a method of isolating nucleic acids from cells according to one embodiment of the present invention;

In the method of isolating nucleic acids from cells according to the current embodiment of the present invention, CNTs and silica beads are introduced into a sample solution containing the cells. CNTs are in the form of tubes wherein a carbon atom binds to other carbon atoms to form a hexagonal honeycomb. CNTs are very small materials having a diameter of the order of nanometers. Methods of synthesizing a large amount of CNTs are known in the art, including, for example, an arc-discharge method, a laser vaporization method, a plasma enhanced chemical vapor deposition method, a thermal chemical vapor deposition method, a vapor phase growth method, an electrolytic method, and a flame synthesis method. Additional methods of synthesizing a large amount of CNTs are known and available to one of ordinary skill in the art. CNTs have excellent mechanical properties, electrical selectivity, excellent field emission properties, and high efficiency of hydrogen storage capacity, etc.

According to the current embodiment, the method of isolating nucleic acids from cells, the sample solution containing cells, CNTs and silica beads is irradiated with a laser beam to disrupt the cells, releasing the nucleic acids from the disrupted cells. Once the nucleic acids have been released from the disrupted cells, the nucleic acids bind to the silica beads. Without being held to theory, it is believed that when the CNTs are irradiated with the laser beam, internal energy is converted to heat due to an intrinsic excitation frequency of CNTs and the effects of Raman resonance and electronic polarization. When the CNTs in solution are irradiated with a large portion of energy of light at a specific wavelength, for example, a wavelength of 808 nm, the temperature of the solution can increase to about 100° C. As a result of the increased temperature of the solution, disruption of cells and release of genomic DNA molecules can be efficiently achieved.

It should be noted that CNTs have a high affinity to adsorb nucleic acids. However, the affinity of CNTs to bind nucleic acid is too high for efficient isolation of nucleic acids using CNTs alone. In particular, large portions of the released nucleic acids are adsorbed on the CNTs. Due to the a high affinity of CNTs to adsorb nucleic acids it is nearly impossible to isolate the nucleic acids bound to the CNTs.

Silica beads are clear and cannot absorb a laser beam. As a result, irradiating a sample solution, which contains only silica beads, cannot disrupt the cells in the solution. However, the silica beads have a higher binding efficiency for the nucleic acids than the CNTs and thus, may bind to the released nucleic acids. Unlike the CNTs, nucleic acids bound to the silica beads can be efficiently removed, or eluted, from the silica beads. Thus, silica beads can be used to isolate the nucleic acids. That is, according to one embodiment, the laser beam irradiates the CNTs, increasing the temperature of the solution, which disrupts the cells in the sample solution. In addition, the silica beads adsorb the release nucleic acids, which can then be efficiently isolated from the silica beads.

According to the current embodiment of the present invention, the laser can be a pulse laser or a continuous wave laser. The effects of laser ablation cannot be efficiently induced when the power of the laser is too low. With respect to the power of the laser, a pulse laser should deliver a pulse of greater than or equal to about 1 mJ/pulse. The pulse laser can have a power of greater than or equal to about 3 mJ/pulse. If the power of the pulse laser is less than about 1 mJ/pulse, sufficient energy to disrupt the cells is not delivered to the cells. With respect to the power of the laser, a continuous wave (CW) laser should deliver greater than or equal to about 10 mW. The CW laser may have a power of greater than or equal to about 100 mW. If the power of the CW laser is less than about 10 mW, sufficient energy to disrupt the cells is not delivered to the cells.

In the method of isolating nucleic acids from cells according to the current embodiment of the present invention, the laser beam should be generated in a specific wavelength band of which energy the CNTs may absorb. The laser beam may be generated in a wavelength band of greater than or equal to about 400 nm, preferably a wavelength band of about 750-1300 nm. If the laser beam is generated in a wavelength of less than about 400 nm, DNA molecules may be denatured or damaged. Further, the laser beam can be generated in one or more wavelength bands. That is, the laser beam can emit a single wavelength or emit two different wavelengths within the range of the above wavelength bands.

In the method of isolating nucleic acids from cells according to the current embodiment of the present invention, a nucleic acid eluting solution can be added to the silica beads to which the nucleic acids are bound, to elute the nucleic acids from the silica beads. The nucleic acids isolated from the silica beads following addition of the nucleic acid eluting solution to the silica beads can then be subsequently used for various applications, including, for example, amplification of the nucleic acids.

In one embodiment, the method of isolating nucleic acids from cells may further comprise the process of amplifying the eluted nucleic acids. Methods of amplifying DNA are known and routinely used by one of ordinary skill in the art. Most suitably, a polymerase chain reaction (PCR) method can be used to sufficiently amplify the eluted DNA molecules. Other methods of amplifying DNA can be used, including, for example, a real-time PCR method.

In an exemplary embodiment, the silica beads can have a diameter of about 50 nm to about 1,000 μm, preferably about 1 to about 50 μm. If the diameter of the silica beads is less than about 50 nm, the silica beads may be vulnerable to physical and mechanical impact. If the diameter of the silica beads is greater than about 1,000 μm, they cannot be used in a small-sized lab-on-a-chip (LOC). The silica beads may be a mixture of silica beads having two or more sizes. That is, the silica beads may have an identical size or different sizes.

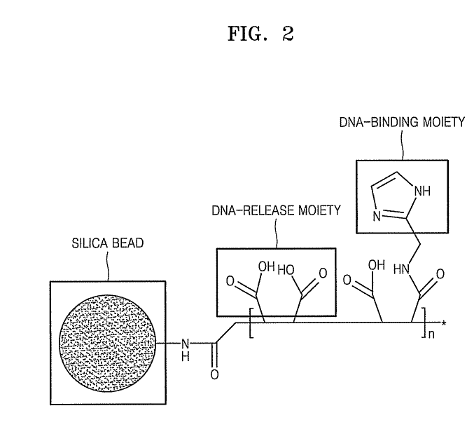

In the method of isolating nucleic acids from cells according to the current embodiment of the present invention, the silica beads comprise a surface functional group. In one embodiment, the silica beads have a surface functional group comprising both a DNA-binding moiety and a DNA-release moiety. FIG. 2 is a schematic diagram illustrating a silica bead suitable for use in the method of isolating nucleic acids from cells described with reference to FIG. 1. Referring to FIG. 2, the surface of the silica bead has a DNA-binding moiety and a DNA-release moiety. In one embodiment, the DNA-binding moiety is a positive ionizable functional group and, at an acidic pH (pH 3-5), it is positively charged. At an acidic pH (pH 3-5), the DNA-binding moiety is positively charged and thus, can bind to a negatively charged nucleic acid molecule, via electrostatic attraction. The DNA-release moiety is a negative ionizable functional group and, at a basic pH (pH 7-9), it is negatively charged. At a basic pH (pH 7-9) the DNA-binding moiety is negatively charged and thus, repels the negatively charged nucleic acid by electrostatic repulsion. The DNA-binding moiety can be an aromatic heterocyclic amine and the DNA-release moiety may be an organic acid. The aromatic heterocyclic amine can be imidazole, pyridine, pyrrole, or the like, but the present invention is not limited thereto. The organic acid can be a carboxylic acid, or the like, but the present invention is not limited thereto.

In the method of isolating nucleic acids from cells according to the current embodiment of the present invention, the silica beads introduced to the sample solution can be added directly or added in the form of a solution, preferably the silica beads are introduced to the sample solution in the form of a solution. The solution containing the silica beads can have a pH of 3-5. If the pH of the solution is outside a range of pH 3-5, the efficiency of the nucleic acids to bind to the silica beads can decrease.

In one embodiment of the method of isolating nucleic acids from cells, the CNTs may comprise single-wall nanotubes, multi-wall nanotubes, rope nanotubes, or combinations comprising at least one of the foregoing nanotubes. CNTs are in the form of long and fine tubes having a diameter of several nanometers. CNTs may be formed of a conductive material or a semiconductive material according to their unique shape and rolled structure. Due to their unique electrical properties, CNTs have been of growing interest. CNTs can be classified into two types, i.e., single-wall nanotubes and multi-wall nanotubes. Single-wall nanotubes consist of a single wall and multi-wall nanotubes consist of a plurality of walls. Single-wall nanotubes are more flexible than multi-wall nanotubes and thus, have a tendency to form a rope consisting of several nanotubes, i.e., rope-nanotubes.

The CNTs can be impregnated with platinum, gold, ruthenium, silver, nickel, copper, chromium, or palladium, and the like, or combinations comprising at least one of the foregoing. In the current embodiment, bare CNTs or CNTs impregnated with platinum, gold, ruthenium, silver, nickel, copper, chromium, or palladium, and the like, or combinations comprising at least one of the foregoing can be used.

In the method of isolating nucleic acids from cells according to the current embodiment of the present invention, the nucleic acid eluting solution may have a pH of 7-9. If the pH of the nucleic acid eluting solution is less than 7, the elution efficiency of the nucleic acids decreases. If the pH of the nucleic acid eluting solution is greater than 9, a subsequent process, such as, the process of DNA amplification, may be adversely affected. The nucleic acid eluting solution can include phosphates, acetates, citrates, Tris, sulfates, etc., but the present invention is not limited thereto. The concentration of the nucleic acid eluting solution may be about 10 nm to about 1000 mM. If the concentration of the nucleic acid eluting solution is outside the above range, the elution efficiency of the nucleic acids can decrease and a subsequent process, such as, for example, the process of DNA amplification, may be adversely affected.

According to another embodiment of the present invention, there is provided an apparatus for continuously performing isolation and amplification of nucleic acids, comprising, a cell disruption micro-chamber having a sample inlet through which a sample solution containing cells, CNTs, and silica beads are introduced; a sample storage unit being in a fluid communication with the cell disruption micro-chamber through a micro-channel and supplying the sample solution containing the cells, CNTs, and silica beads to the cell disruption micro-chamber through the micro-channel and; a laser generation unit attached to the cell disruption micro-chamber and supplying a laser beam to the cell disruption micro-chamber; a PCR mixture storage unit being in a fluid communication with the cell disruption micro-chamber through a micro-channel and supplying a PCR mixture to the cell disruption micro-chamber through the micro-channel; and a heating and cooling unit heating and cooling the cell disruption micro-chamber.

The sample solution containing cells can be introduced into the cell disruption micro-chamber through the sample inlet. The introduced sample solution is mixed with CNTs and silica beads. The introduced sample solution can be uniformly mixed with the CNTs and silica beads using a vibrator. Examples of the vibrator include a sonicator, a vibrator using a magnetic field, a vibrator using an electric field, a mechanical vibrator such as vortex, or a piezoelectric material. The vibrator can be attached to the cell disruption micro-chamber and can be any device that can vibrate a mixture solution containing the cells, the CNTs, and the silica beads. In one embodiment, the sample can be irradiated with a laser beam while the sample solution is continuously vibrated.

In one embodiment, the cell disruption micro-chamber comprises a window made of a material through which the laser beam can sufficiently pass. When the CNTs are irradiated with the laser beam, the CNTs convert the light into heat, thereby increasing the temperature of the sample solution to about 100° C.

In one embodiment, the apparatus comprises a PCR mixture storage unit being in a fluid communication with the cell disruption micro-chamber through the micro-channel. The PCR storage unit can supply a PCR mixture to the cell disruption micro-chamber through the micro-channel.

In another embodiment, the apparatus comprises a heating and cooling unit. The heating and cooling unit serves the function of heating and cooling the cell disruption micro-chamber. The heating and cooling unit can be attached to the cell disruption micro-chamber and can be any device capable of heating and cooling the cell disruption micro-chamber.

A system or method for amplifying isolated DNA molecules is necessary to realize a LOC. Most suitably, a PCR method can be used to sufficiently amplify the eluted DNA molecules. Other methods of amplifying DNA molecules can be used. For example, a real-time PCR method can also be used. Thus, according to the current embodiment, the apparatus comprises the PCR mixture storage unit and the heating and cooling unit, thereby allowing the isolation and the amplification of the nucleic acids to be continuously performed.

According to one embodiment, the apparatus, the laser generation unit of the apparatus can produce a pulse laser beam or CW laser beam. The effects of laser ablation cannot be efficiently induced when the power of the laser generation unit is too low. With respect to the power of the laser, a pulse laser should deliver a pulse of greater than or equal to about 1 mJ/pulse. The pulse laser can have a power of greater than or equal to about 3 mJ/pulse. If the power of the pulse laser is less than about 1 mJ/pulse, sufficient energy to disrupt the cells is not delivered to the cells. With respect to the power of the laser, a continuous wave (CW) laser should deliver greater than or equal to about 10 mW. The CW laser may have a power of greater than or equal to about 100 mW. If the power of the CW laser is less than about 10 mW, sufficient energy to disrupt the cells is not delivered to the cells.

In one embodiment, the pulse laser beam or CW laser beam should be generated in a specific wavelength band of which energy the CNTs may absorb. The pulse laser beam, or the CW laser beam may be generated in a wavelength band of greater than or equal to about 400 nm, preferably a wavelength band of about 750 nm to about 1300 nm. If the pulse laser beam, or the CW laser beam is generated in a wavelength of less than about 400 nm, DNA molecules may be denatured or damaged. Further, the pulse laser beam or CW laser beam can be generated in one or more wavelength bands. That is, the pulse laser beam, or the CW laser beam can emit a singe wavelength or two different wavelengths within the range of the above wavelength bands.

According to another embodiment of the present invention, there is provided a LOC comprising the apparatus, which can continuously perform isolation and amplification of nucleic acids according to the previous embodiment of the present invention. Each of the functional components of the apparatus for the isolation and amplification of the nucleic acids may be embodied in a process-on-a-chip and further, in a LOC, using a known technique of microfluidics and a microelectromechanical system (MEMS) device.

The present invention will be described in further detail with reference to the following examples. These examples are for illustrative purposes only and are not intended to limit the scope of the invention.

### EXAMPLES

### Example 1

### Synthesis of CNTs

In the current Example, bare CNTs and Pt-impregnated CNTs were synthesized. The bare CNTs were CE601B (available from CNI, U.S.A.), which were synthesized using a chemical vapor deposition (CVD) method and consisted of 1-3 CNT walls. The Pt-impregnated CNTs were synthesized as follows. 0.25 g of bare CNTs was added to 100 mL of distilled water and 80 mL of ethylene glycol and the mixture was subjected to an ultrasonic dispersion. Then, the resultant product was mixed with 20 mL of a solution of a Pt precursor, H₂PtCl₆, in ethylene glycol, and the mixture was refluxed at 110° C. to reduce the Pt precursor to nano-sized platinum. The Pt-impregnated CNTs were washed several times with distilled water using a centrifuge and dried using a freeze dryer.

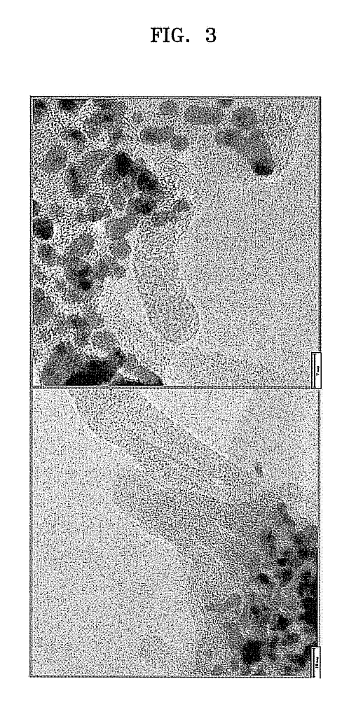

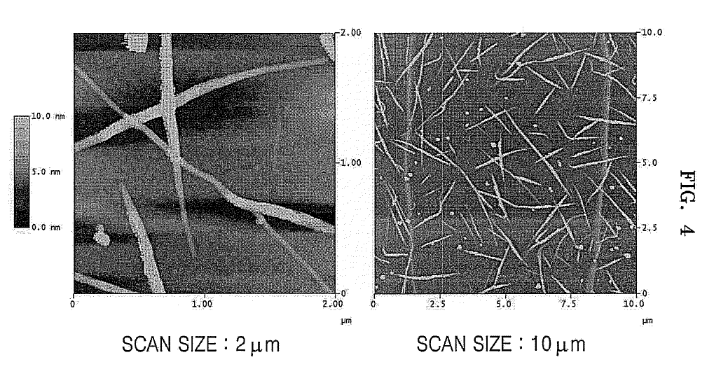

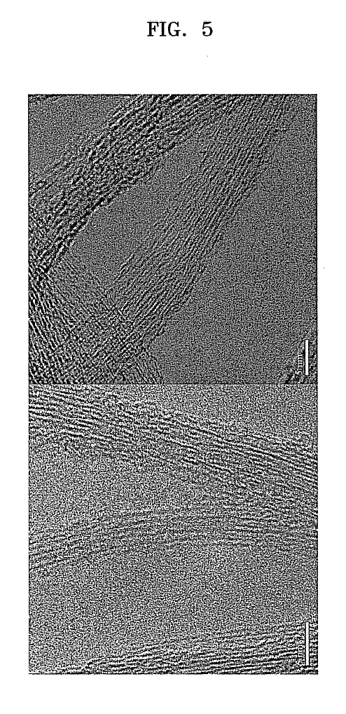

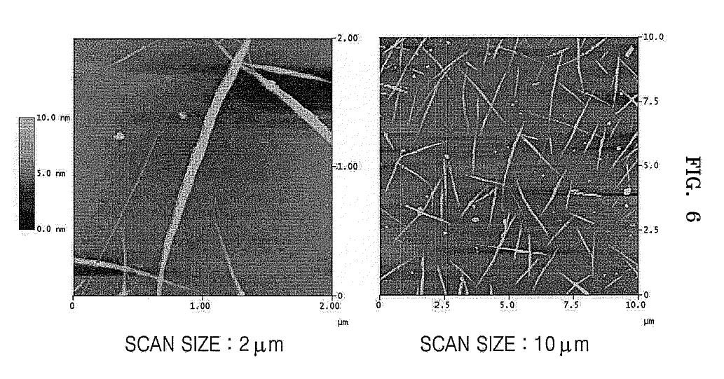

The purity and shape of the bare CNTs and Pt-impregnated CNTs were examined using a transmission electron microscope (TEM) and an atomic force microscope (AFM). FIGS. 3 through 6 are photographic images illustrate the bare CNTs and the Pt-impregnated CNTs described above. In particular, FIG. 3 shows a photographic representation of transmission electron microscope (TEM) images of Pt-impregnated carbon nanotubes (CNTs). FIG. 4 shows photographic representation of atomic force microscope (AFM) images of Pt-impregnated CNTs. FIG. 5 shows a photographic representation of TEM images of bare CNTs. FIG. 6 shows a photographic representation of AMF images of bare CNTs.

Referring to FIG. 3, it can be confirmed that the Pt-impregnated CNTs are surrounded by amorphous carbon, and the concentration of Pt in the CNTs was 46.465% by weight. Referring to FIG. 5, it can be confirmed that single-wall nanotubes are present in the form of ropes and the ropes are surrounded by a small amount of amorphous carbon and not contaminated by other metals. Referring to FIGS. 4 and 6, it can be confirmed that the length of the CNTs is about 1-5 μm.

### Example 2

### The Effects of Laser Irradiation on Temperature

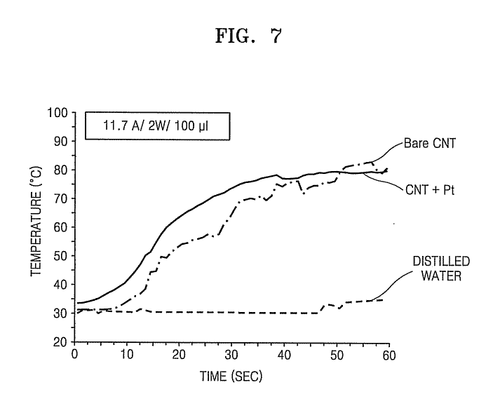

In the current example, the effect of laser irradiation on the temperature of a solution was examined. For this example, a laser of 11.7 A and 2 W irradiated 100 μl of each of a solution containing bare CNTs in distilled water; a solution containing Pt-impregnated CNTs in distilled water; and distilled water. FIG. 7 is a graph illustrating laser irradiation time vs. temperature of a solution. Referring to FIG. 7, it can be confirmed that the temperature of distilled water hardly increased following irradiation. For the solution containing bare CNTs and the solution containing Pt-impregnated CNTs the temperature increased to 80° C. or greater due to the laser irradiation for about one minute. Thus, it can be confirmed that CNTs are very useful for increasing the temperature of a solution by absorbing a laser beam, and thus cells can be efficiently disrupted using CNTs.

### Example 3

### The Effects of Laser Irradiation and CNTs on Cell Disruption

The following example examined the effects of laser irradiation and CNTs on cell disruption. In particular, a sample solution containing E. coli strain BL21 and silica beads, together with the bare CNTs, or together with the Pt-impregnated CNTs described in Example 1, was subjected to laser irradiation. The solution was irradiated with a laser beam at a wavelength of 808 nm with a power of 2 W for 60 seconds. After the laser irradiation, E. coli cells were collected from the sample solution by centrifuging the irradiated cells at 5000 rpm for 2 minutes. The collected cells were then rinsed twice with 3 ml of a phosphate buffered saline (PBS) solution. The cells were then resuspended in phosphate buffered saline solution (PBS) (cell density; 1×10⁵ cells/μl). Cell viability was then determined. The number of viable cells was determined by the ability of single cells to form colonies. Aliquots of E. coli cells (1×10³) collected after the irradiation were spread on BHI plates. The plates were incubated at 37° C. overnight and the number of colonies was counted.

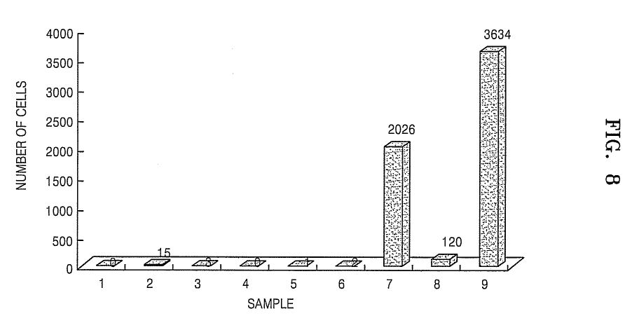

FIG. 8 is a graph illustrating the number of E. coli cells after laser irradiation with or without CNTs. Referring to FIG. 8, symbols 1 through 3 in the bottom of the graph represent a sample solution containing the E. coli cells and CNTs, which was subjected to laser irradiation (repeated 3 times); symbols 4 through 6 in the bottom of the graph represent a sample solution containing the E. coli cells and Pt-impregnated CNTs, which was subjected to laser irradiation (repeated 3 times); symbol 7 represents a sample solution E. coli cells not including CNTs which was subjected to laser irradiation; symbol 8 represents a sample E. coli cells not including CNTs, which subjected to boiling at 95° C. for 5 minutes; and symbol 9 represents a sample solution which did not include CNTs which was not subjected to laser irradiation.

In case of the sample solution containing the E. coli cells and CNTs, which was subjected to laser irradiation (symbols 1 through 3) and the sample solution containing the E. coli cells and Pt-impregnated CNTs and subjected to laser irradiation (symbols 4 through 6), most E. coli cells were disrupted. However, in case of the sample solution containing the E. coli cells not including CNTs, which was subjected to laser irradiation (symbol 7), the E. coli cells were hardly disrupted. The results of the sample solution containing the E. coli cells not including CNTs, which was subjected to laser irradiation (symbol 7) is similar to the sample solution containing the E. coli cells not including CNTs, which was not subjected to laser irradiation (symbol 9). Sample solutions containing E. coli cells and including bare CNTs or Pt-impregnated CNTs, not subjected to laser irradiation, “CNT Alone” and “CNT+Pt Alone”, respectively, provided results similar to the sample solution containing the E. coli cells not including CNTs, which was not subjected to laser irradiation. (data not shown) In case of the sample subjected to boiling at 95° C. for 5 minutes, most E. coli cells were also disrupted.

Thus, as demonstrated by FIG. 8, it can be confirmed that when using CNTs (or Pt-impregnated CNTs) and laser irradiation according to an embodiment of the present invention, cells can be efficiently disrupted.

### Example 4

### Amplification of Nucleic Acids Released from Cells Disrupted by Laser Irradiation and CNTs

For this Example, PCR amplification was performed to quantify the amount of DNA released from the E. coli cells disrupted in Example 3. The following pair of PCR primers was used: forward primer (5′-cccagactcc tacgcgaggc-3′: SEQ ID NO: 1) and reverse primer (5′-gtattaccgc aactgctggc ac-3′: SEQ ID NO: 2). These primers are complementary to respective ends of a gene encoding 16S ribosomal RNA and allow amplification of the entire coding region during PCR.

2 μl of each of the final solutions obtained in Example 3 was added to a mixture of 2 μl of Solgent™ PCR buffer (10×), 2 μl at of MgCl₂ (25 mM), 2 μl of 2 mM dNTP, 1 μl of Taq™ polymerase (Solgent™) 0.6 U/μl, 1 μl of each of 10 pM forward and reverse primers, and 9 μl of triple distilled water and they were uniformly mixed to obtain a PCR mixture (a total volume of 20 μl). The obtained PCR mixture was subjected to a PCR using TMC-1000. The conditions of the PCR were as follows: initial denaturation at 95° C. for 1 minute and 25 cycles with each cycle including denaturation at 95° C. for 5 sec, annealing at 60° C. for 13 sec and extension at 72° C. for 15 sec. The amplified DNA molecules were analyzed in an Agilent BioAnalyzer™ 2100 (Agilent Technologies, Palo Alto, Calif.) using a set of reagents of DNA 500 assay, which is commercially available.

Table 1 shows the concentrations of the PCR products from the DNA obtained in Example 3. Referring to Table 1, “Boiling” represents a sample subjected to boiling at 95° C. for 5 minutes (positive control); “CNT Alone” and “CNT+Pt Alone” represent sample solutions containing E. coli cells and including bare CNTs or Pt-impregnated CNTs, respectively, and not subjected to laser irradiation; “CNT+Pt+Laser” and “CNT+Laser” represent sample solutions containing E. coli cells and including Pt-impregnated CNTs or bare CNTs, respectively, and subjected to laser irradiation according to an embodiment of the present invention; “Laser Alone” represents a sample solution containing E. coli cells and not including CNTs and subjected to laser irradiation; “Laser+Silica bead” represents a sample including silica beads (without CNTs) and subjected to laser irradiation.

TABLE 1

| | |
|---|---|
| Sample | DNA concentration (ng/μl) |

| | | |
|---|---|---|
| 1. | Boiling | 26.7 |
| 2. | CNT Alone | 0.00 |
| 3. | CNT + Pt Alone | 0.00 |
| 4. | CNT + Pt + Laser | 4.2 |
| 5. | CNT + Pt + Laser | 3.8 |
| 6. | CNT + Pt + Laser | 4.2 |
| 7. | Laser Alone | 0.00 |
| 8. | Laser + Silica Bead | 0.00 |
| 9. | CNT + Laser | 31.35 |
| 10. | CNT + Laser | 30.2 |
| 11. | CNT + Laser | 31 |

Referring to Table 1, it can be confirmed that the results in Example 4 are similar to the results in FIG. 8. That is, in case of “CNT Alone”, “CNT+Pt Alone”, “Laser Alone” and “Laser+Silica bead”, the cells were not disrupted and the DNA molecules were not efficiently released from the cells, and as a result, PCR products were not produced. In case of “CNT+Pt+Laser” and “CNT+Laser” according to an embodiment of the present invention, many cells were disrupted and a large amount of DNA was released, and as a result, a large amount of PCR product was produced. In case of “Laser+Silica Bead”, PCR products were not produced, since the silica beads cannot absorb the laser beam and thus the cells were not disrupted.

As described above, in the method of isolating nucleic acids from cells or viruses according to the present invention, disruption of cells, extraction of nucleic acids, and amplification of nucleic acids can be performed in a single chamber, thereby allowing the use of a small apparatus. Further, the disruption of cells and the extraction of nucleic acids can be performed within one minute and thus, isolation time of nucleic acids can be greatly shortened.

While certain parameters were used in experiments described herein, it is recognized that many other values for the parameters can be utilized to find an optimal set of parameters for each different type of cell. The cells utilized in the experiments consisted of gram negative bacteria. It is anticipated that all different types of cells and virus particles may be utilized and parameters defined therefore. The same method and apparatus to determine the parameters is clearly applicable.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of isolating nucleic acids from cells comprising:
introducing carbon nanotubes (CNTs) and silica beads into a sample solution containing the cells;
irradiating the sample solution with a laser beam to disrupt the cells, releasing nucleic acids from the disrupted cells, wherein the nucleic acids bind to the silica beads; and
introducing a nucleic acid-eluting solution to the silica beads to which the nucleic acids are bound, to elute the nucleic acids from the silica beads.

2. The method of claim 1, further comprising amplifying the eluted nucleic acids.

3. The method of claim 1, wherein the solution is irradiated by a pulse laser or a continuous wave (CW) laser.

4. The method of claim 3, wherein the pulse laser has a power of greater than or equal to 1 mJ/pulse and the continuous wave laser has a power of greater than or equal to 10 mW.

5. The method of claim 4, wherein the pulse laser has a power of greater than or equal to 3 mJ/pulse and the continuous wave laser has a power of greater than or equal to 100 mW.

6. The method of claim 1, wherein the laser beam is generated in a wavelength band of greater than or equal to 400 nm.

7. The method of claim 6, wherein the laser beam is generated in a wavelength band of 750-1300 nm.

8. The method of claim 6, wherein the laser beam is generated in one or more wavelength bands.

9. The method of claim 1, wherein the silica beads have a diameter of about 50 nm to about 1,000 μm.

10. The method of claim 1, wherein the silica beads comprise one or more surface functional group having both a DNA-binding moiety and a DNA-release moiety.

11. The method of claim 10, wherein the DNA-binding moiety is an aromatic heterocyclic amine and the DNA-release moiety is an organic acid.

12. The method of claim 11, wherein the aromatic heterocyclic amine is imidazole, pyridine, or pyrrole and the organic acid is a carboxylic acid.

13. The method of claim 1, wherein the solution containing the silica beads has a pH of 3-5.

14. The method of claim 1, wherein the CNTs are single-wall nanotubes, multi-wall nanotubes, or rope nanotubes.

15. The method of claim 1, wherein the CNTs are impregnated with platinum, gold, ruthenium, silver, nickel, copper, chromium, palladium, or a combination comprising at least one of the foregoing metals.

16. The method of claim 1, wherein the nucleic acid eluting solution has a pH of 7-9.

17. The method of claim 1, wherein the sample solution containing the cells is saliva, urine, blood, serum, or a cell culture.

18. An apparatus for continuously performing isolation and amplification of nucleic acids, comprising:
a cell disruption micro-chamber having a sample inlet through which a sample solution containing cells, CNTs, and silica beads are introduced;
a sample storage unit being in a fluid communication with the cell disruption micro-chamber through a micro-channel and supplying the sample solution containing the cells, CNTs, and silica beads to the cell disruption micro-chamber through the micro-channel. and
a laser generation unit attached to the cell disruption micro-chamber wherein the laser generation unit can irradiate the cell disruption micro-chamber with a laser beam; and
a polymerase chain reaction (PCR) mixture storage unit being in a fluid communication with the cell disruption micro-chamber through a micro-channel, wherein the polymerase chain reaction (PCR) mixture storage unit can supply a PCR mixture to the cell disruption micro-chamber through the micro-channel; and
a heating and cooling unit, wherein the heating and cooling unit can heat or cool the cell disruption micro-chamber.

19. The apparatus of claim 18, wherein the laser generation unit irradiates the cell disruption micro-chamber using a pulse laser or a continuous wave laser.

20. The apparatus of claim 19, wherein the pulse laser has a power of greater than or equal to 3 mJ/pulse and the continuous wave laser has a power of greater than or equal to 100 mW.

21. The apparatus of claim 18, wherein the laser beam is generated in a wavelength band of greater than or equal to 400 nm.

22. The apparatus of claim 21, wherein the laser beam is generated in a wavelength band of 750-1300 nm.

23. The apparatus of claim 18, wherein the laser beam is generated in one or more wavelength bands.

24. A lab-on-a-chip (LOC) comprising the apparatus of claim 18.


SEQUENCE LISTING (2 sequences)

SEQ ID NO: 1 - DNA, 20 bp, Artificial Sequence
  forward primer
cccagactcctacgcgaggc

SEQ ID NO: 2 - DNA, 22 bp, Artificial Sequence
  reverse primer
gtattaccgcaactgctggcac